(19) United States
(12) Patent Application Publication
(10) Pub. No.: US 2022/0091175 A1
(43) Pub. Date: Mar. 24, 2022

(54) SEMICONDUCTOR PRODUCT GRADING METHOD AND GRADING SYSTEM

(71) Applicant: Changxin Memory Technologies, Inc., Hefei City (CN)

(72) Inventor: Chia-Sheng LIN, Hefei City (CN)

(21) Appl. No.: 17/451,343

(22) Filed: Oct. 19, 2021

Related U.S. Application Data
(63) Continuation of application No. PCT/CN2021/103231, filed on Jun. 29, 2021.

(30) Foreign Application Priority Data
Sep. 24, 2020 (CN) 202011015063.6

(51) Int. Cl. G01R 31/26; G01R 31/28

(52) U.S. Cl. CPC G01R 31/2601; G01R 31/2846; G01R 31/2834

(57) ABSTRACT

The present application discloses a semiconductor product grading method and grading system. The grading method includes: at the time of an electric property test, classifying the several semiconductor products into a plurality of groups according to several test parameters obtained from the test; carrying out a principal component analysis on the corresponding several test parameters in each group, to obtain eigen parameters for each group; and determining, based on the eigen parameters, grades for the semiconductor products in each group. The semiconductor products in each group are graded through several eigen parameters with similar attributes, so the accuracy of the existing semiconductor product grading is improved and the semiconductor products that meet different customer requirements can be accurately and quickly screened out for shipping.

### CROSS-REFERENCE TO RELATED APPLICATIONS

This application is a continuation application of International Patent Application No. PCT/CN2021/103231, filed on Jun. 29, 2021, which claims priority to Chinese Patent Application No. 202011015063.6, filed with the Chinese Patent Office on Sep. 24, 2020 and entitled “SEMICONDUCTOR PRODUCT GRADING METHOD AND GRADING SYSTEM.” International Patent Application No. PCT/CN2021/103231 and Chinese Patent Application No. 202011015063.6 are incorporated herein by reference in their entireties.

### TECHNICAL FIELD

The present invention relates to the field of semiconductor testing, and in particular to a semiconductor product grading method and grading system.

### BACKGROUND

Integrated circuits are a type of micro electronic devices or components. According to such integrated circuits, by utilizing semiconductor manufacturing processes such as oxidation, photoetching, diffusion, epitaxy, masking, sputtering, etc., elements such as transistors, resistors, capacitors, inductors or the like as well as wirings, which are required for a circuit, are interconnected and then fabricated on one or several semiconductor wafers or dielectric substrates, followed by being encapsulated within a package to attain a microstructure or chip having desired circuit functions.

Upon completion of the fabrication, integrated circuits and chips are shipped to corresponding customers. However, it is impossible to have a grasp of the advantages and disadvantages of various parameters of every chip during the fabrication process of the integrated circuits. As a result, once these integrated circuits and chips are shipped to some customers, the risk of returns is increased since potential quality-related issues that cannot be easily handled may occur.

Now, in order to address the foregoing problem, dies or chips are typically tested before their shipping. Defective or unqualified products that are manufactured during the manufacturing process are picked out by means of the testing, or the property parameters of the dies or chips are learnt from the testing and subsequently the grades of the products are empirically determined on the basis of some parameters resulting from the testing. For example, determination for the grades of the products relies upon speed parameters obtained from the testing (products with high speed parameters have higher grades and products with low speed parameters have lower grades). Such an existing grading method for product grading, however, cannot provide an accurate product grading, and accordingly how to provide a more accurate grading for products is still an urgent problem to be solved.

### SUMMARY

The technical problem to be solved by the present application is how to provide a more accurate grading for products.

Provided in the present application is a semiconductor product grading method, which includes:
providing several semiconductor products of a same lot;
carrying out electric property tests on the several semiconductor products to obtain several test parameters;
classifying the several semiconductor products into a plurality of groups according to the several test parameters;
carrying out a principal component analysis on the corresponding several test parameters in each group, to obtain eigen parameters for each group; and
determining, based on the eigen parameters, grades for the semiconductor products in each group.

Also provided in the present application a semiconductor product grading system, which includes:
a testing unit, configured to carry out electric property tests on several semiconductor products of a same lot, to obtain several test parameters;
a classifying unit, configured to classify the several semiconductor products into a plurality of groups according to the several test parameters;
an eigen parameter obtaining unit, configured to carry out a principal component analysis on the corresponding several test parameters in each group, to obtain eigen parameters for each group; and
a grading unit, configured to determine, based on the eigen parameters, grades for the semiconductor products in each group.

### BRIEF DESCRIPTION OF DRAWINGS

FIG. 1 to FIG. 3 are schematic flow charts of a semiconductor product grading method according to the embodiments of the present application;

FIG. 4 to FIG. 9 are schematic structural diagrams of a semiconductor product grading process according to the embodiments of the present application; and

FIG. 10 is a schematic structural diagram of a semiconductor product grading system according to the embodiments of the present application.

### DESCRIPTION OF EMBODIMENTS

In order to provide a better clarity of the objects, technical solutions, and advantages of the present application, the present application will be further described below in details in conjunction with the specific embodiments and with reference to the accompanying drawings. However, it shall be appreciated that these descriptions are exemplary only, and are not intended to limit the scope of the present application. In addition, descriptions of well-known structures and technologies are omitted in the following description so as to avoid unnecessarily obscuring the concept of the present application.

As mentioned in the Background, such an existing grading method for product grading cannot provide an accurate product grading.

It has been found in researches that currently, product grades are usually dictated by a single test parameter. Yet, there are nearly thousands of test parameter types during a process of fabrication of integrated circuits, and different customers have different requirements for the property parameters of products. Accordingly, such an existing grading method hardly offers an accurate reflection of product grades.

To this end, the present application provides a semiconductor product grading method and grading system. The grading method includes: at the time of an electric property test, classifying several semiconductor products into a plurality of groups according to several test parameters obtained from the test; carrying out a principal component analysis on the corresponding several test parameters in each group, to obtain eigen parameters for each group; and determining, based on the eigen parameters, grades for the semiconductor products in each group. The semiconductor products in each group are graded through several eigen parameters with similar attributes, so the accuracy of the existing semiconductor product grading is improved and the semiconductor products that meet different customer requirements can be accurately and quickly screened out for shipping.

In order to make the above objects, features and advantages of the present application more apparent and understandable, the specific implementations of the present application will be described below in detail with reference to the accompanying drawings. When describing the embodiments of the present application in detail, the schematic diagrams attached hereto, for illustrative purposes, are not partially enlarged based on the regular scale, and are not intended to limit the protection scope of the present application but only serve as examples. Besides, the three-dimensional size of length, width and depth should be made clear in practical application.

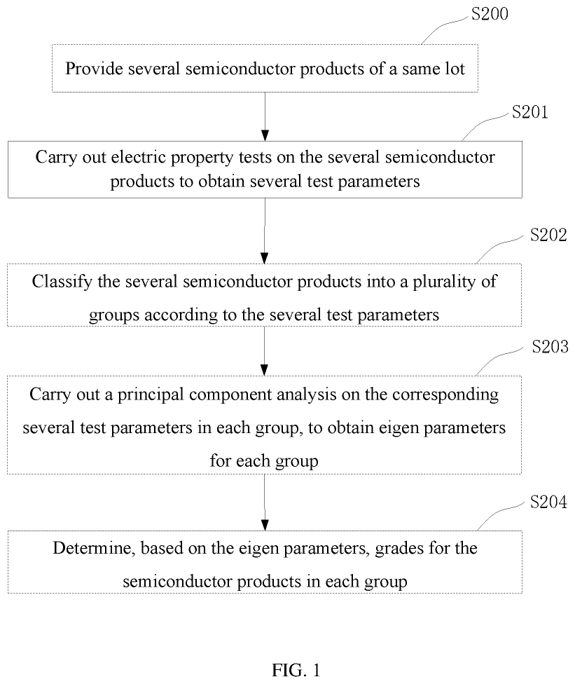

Referring to FIG. 1, provided in an embodiment of the present application is a semiconductor product grading method, which includes:

S200: providing several semiconductor products of a same lot;

S201: carrying out electric property tests on the several semiconductor products to obtain several test parameters;

S202: classifying the several semiconductor products into a plurality of groups according to the several test parameters;

S203: carrying out a principal component analysis on the corresponding several test parameters in each group, to obtain eigen parameters for each group; and

S204: determining, based on the eigen parameters, grades for the semiconductor products in each group.

A detailed description of the foregoing process is given below with reference to the accompanying drawings.

At first, S200 is performed: providing several semiconductor products of a same lot. The semiconductor products, which are chips or dies formed by semiconductor manufacturing processes, may be several semiconductor products on the same wafer, or several semiconductor products on the same group of wafers (i.e., wafers of the same lot).

During actual manufacturing, products are typically represented by lots, one lot includes a plurality of wafers, and wafers of the same lot result in the same product. In particular, for example 1 lot contains 25 wafers, so if 1000 wafers are needed to produce the same product, then there are 40 lots in total.

For the purpose that semiconductor products on each wafer can be tracked and identified during a test process on a production line and thus be readily graded, each semiconductor product will be given a corresponding name, in general these semiconductor products are named in accordance with product names (which may be indicated by A), lots (which may be indicated by L1, L2, . . . , Ln), wafer ordering in a particular lot (which may be indicated by 1, 2, . . . , 25) and ordering of several semiconductor products in a wafer (which may be indicated by D1, D2, . . . , Dn). By way of example, several semiconductor products in the lth wafer of the L1th lot in the product A are named A-L1-1-D1 , A-L1-1-D2, . . . , A-L1-1-Dn.

When a semiconductor process is employed on a production line to fabricate an integrated circuit on a wafer, electric property tests need to be carried out on the semiconductors on the wafer at different stages, wherein the tests include a final test (FT), and intermediate electric property tests carried out before the final test; the final test (FT) is an electric property test subsequent to encapsulation, the intermediate electric property tests are generally electric property tests carried out at different stages of integrated circuit fabrication prior to encapsulation, and the intermediate electric property test needs to be carried out many times. Both the final test (FT) and the intermediate electric property tests need to be carried out in multiple steps in order to test different electric parameters.

In this embodiment, the semiconductor product fabricated on the wafer is a memory, which may specifically be a dynamic random-access memory (DRAM) device. In other embodiments, the semiconductor product fabricated on the wafer is a semiconductor product or integrated circuit having other functions, e.g., sensor, filter circuit, etc.

S201 is performed: carrying out electric property tests on the several semiconductor products to obtain several test parameters.

Typically, electric properties subsequent to encapsulation of the semiconductor product draw the most attention, and electric property parameter during this stage are also parameters listed in product standards. Therefore in this embodiment, the electric property test in this step is the final test carried out on several semiconductor products, and this final test generates several final test parameters.

The test parameters of the semiconductor products at various stages are different from each other, so based on the requirements, the test parameters obtained from the intermediate electric property tests may also become indicators for assessing the properties of the semiconductor products. Thus, in other embodiments, the electric property tests in this step may also be intermediate electric property tests carried out on several semiconductor products, and these intermediate electric property tests generate several intermediate test parameters.

The electric property tests may be carried out on a dedicated test device. In this embodiment, there are three types of test parameters: the first type indicates measurement data parameters (e.g., voltage, current, power, etc.), the second type indicates electrical response speed parameters (e.g., signal transmission rate, etc.), and the third type indicates parameters related to the storage characteristics of a memory (e.g., read characteristics, write characteristics, erase characteristics and the like). In other embodiments, the test parameters may also include other types of test parameters.

During actual performance of the electric property tests, there are dozens of, or even hundreds of electric property parameters in each semiconductor product that need to be tested. With reference to FIG. 4, which is a schematic diagram of several test parameters corresponding to several semiconductor products obtained in an embodiment, each grid in FIG. 4 is indicative of one test parameter for one semiconductor product, and P1, P2, P3, P4, . . . , P300 are indicative of different test parameters, respectively. To clearly illustrate a corresponding relationship between different semiconductor products and their test parameters, each grid in FIG. 4 schematically shows a semiconductor product name, e.g., A-L1-1-D1-P1 indicates a test parameter P1 corresponding to a semiconductor product having a semiconductor product name A-L1-1-D1, and A-L2-1-D2-P2 indicates a test parameter P2 corresponding to a semiconductor product having a semiconductor product name A-L2-1-D2. It shall be noted that the several test parameters shown in FIG. 4 are merely used as an example, and should not limit the protection scope of the present application.

S202 is performed: classifying the several semiconductor products into a plurality of groups according to the several test parameters.

The algorithm utilized to classify the several semiconductor products into a plurality of groups according to the several test parameters is a clustering algorithm. The clustering algorithm involves: splitting, based on a particular standard (e.g., a distance criteria), several data sets into different groups, such that data objects of the same group have as large similarities as possible and also as large differences as possible. That is, following the clustering, the data of the same class gather together as much as possible, and different data are separated from each other as much as possible.

In an embodiment, the clustering algorithm is a K-Means clustering algorithm, a mean shift clustering algorithm, a density-based clustering algorithm, a grid-based clustering algorithm, or a model-based clustering algorithm.

In this embodiment, the process of classifying several semiconductor products into a plurality of groups through use of the K-Means clustering algorithm includes: setting a plurality of different K values, and executing the K-Means clustering algorithm under different K values, to obtain a plurality of corresponding pending groups; obtaining a percentage of inter-group variations of the pending groups under different K values, in a total variation; judging whether a percentage of the inter-group variations corresponding to the pending groups under a certain K value, in the total variation, is within a set threshold range, and if the percentage is within the set threshold range, then taking K pending groups obtained under this K value, as the plurality of groups classified.

In an embodiment, the number of the set K values is 29, and a range for the plurality of different K values is 2 to 30, and thus the K values are 29 natural numbers acquired within the range of 2 to 30. The K-Means clustering algorithm needs to be executed under each K value to obtain a plurality of corresponding pending groups, i.e., for the foregoing 29 K values, the K-Means clustering algorithm needs to be executed 29 times to obtain a plurality of corresponding pending groups; in particular K=2, corresponding to obtaining 2 pending groups; K=3, corresponding to obtaining 3 pending groups; K=4, corresponding to obtaining 4 pending groups; K=5, corresponding to obtaining 5 pending groups; ; K=29, corresponding to obtaining 29 pending groups; and K=30, corresponding to obtaining 30 pending groups.

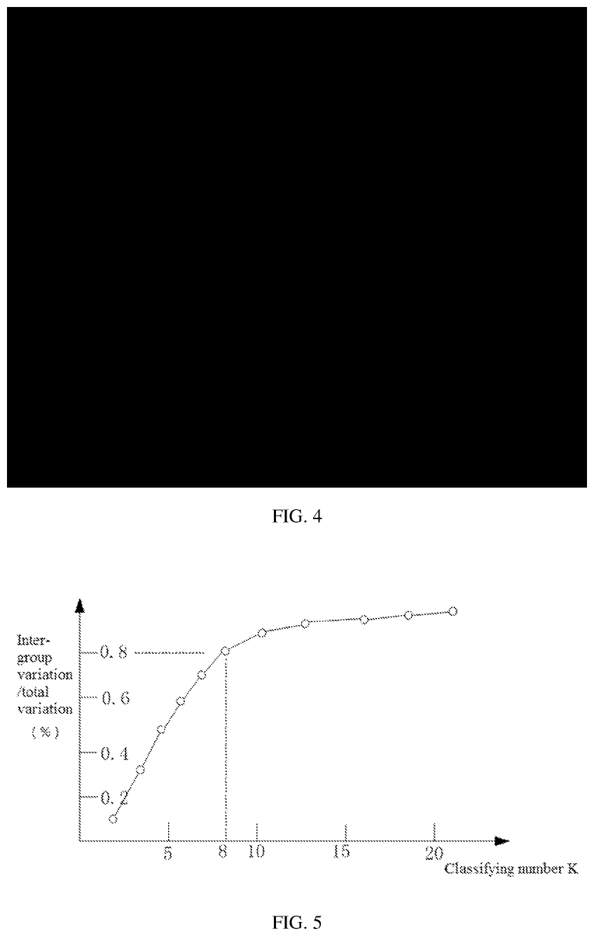

After a plurality of pending groups are obtained, a percentage of inter-group variations of the pending groups under different K values, in a total variation is obtained; it is judged whether a percentage of the inter-group variations corresponding to the pending groups under a certain K value, in the total variation, is within a set threshold range, and if the percentage is within the set threshold range, then K pending groups obtained under this K value are taken as the plurality of groups classified. In particular, by referring to FIG. 5, which is a graph illustrating a tendency that the percentage of the inter-group variations of the pending groups in the total variation changes relative to a particular classifying number K, abscissae indicate the classifying number K and ordinates indicate the percentage of the inter-group variations in the total variation. In this embodiment, the set threshold range is 0.75 to 0.85. It becomes clear from the tendency as shown in FIG. 5 that when K is equal to 8, the corresponding percentage of the inter-group variations in the total variation is 0.8, which is within the set threshold range of 0.75 to 0.85, so the 8 pending groups obtained when K is equal to 8 is taken as the plurality of groups classified. It shall be noted that the tendency as shown in FIG. 5 is merely a schematic illustration, and should not demonstrate the protection scope of the present application. In other embodiment a different threshold range may also be set, so as to obtain a different K value to which the percentage of the inter-group variations in the total variation is corresponding within the threshold value.

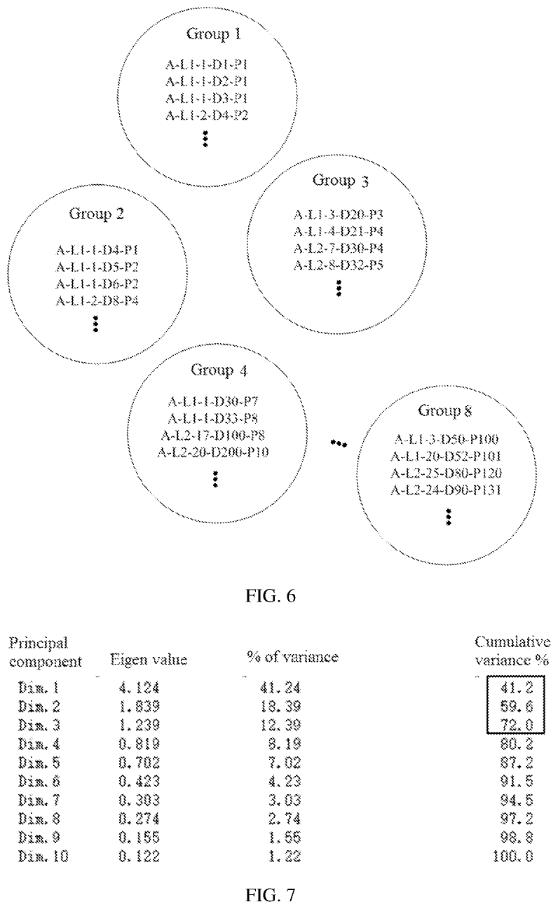

In this embodiment, the number of the groups obtained is 8. Referring to FIG. 6 which is a schematic diagram of 8 groups obtained, group 1 to group 8 are included, each group has several corresponding semiconductor products in which the differences between the test parameters are relatively small, for example, group 1 includes a plurality of semiconductor products and corresponding test parameters, such as A-L1-1-D1-P1, A-L1-1-D2-P1, A-L1-1-D3-P1, A-L1-2-D4-P2, etc. It shall be noted that the number of the groups and the number of the semiconductor products within a particular group in FIG. 6 are an example only, and shall not limit the protection scope of the present application.

S203 is performed: carrying out a principal component analysis on the corresponding several test parameters in each group, to obtain eigen parameters for each group.

In this embodiment, the principal component analysis is carried out on each group to obtain the eigen parameters for each group. As described previously, 8 groups (group 1 to group 8) are obtained and thus the principal component analysis needs to be carried out on the 8 groups to obtain the eigen parameters for each group.

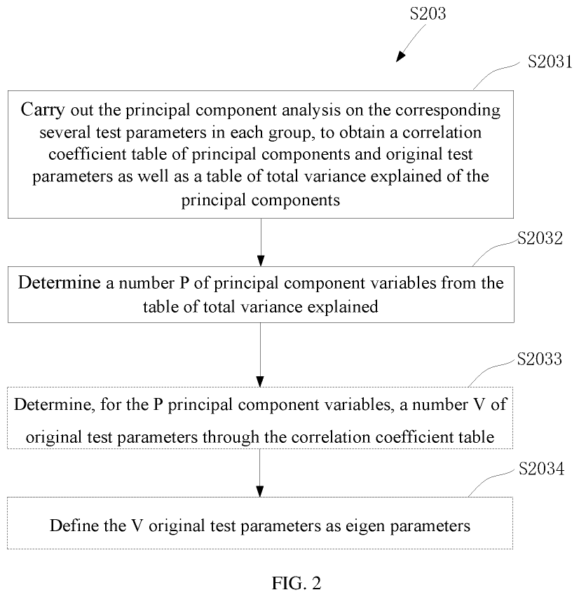

In an embodiment, with reference to FIG. 2, the process of carrying out a principal component analysis on the corresponding several test parameters in each group to obtain main eigen parameters for each group includes: S2031: carrying out the principal component analysis on the corresponding several test parameters in each group, to obtain a correlation coefficient table of principal components and test parameters as well as a table of total variance explained of the principal components; S2032: determining a number P of principal component variables from the table of total variance explained; S2033: determining, for the P principal component variables, a number V of original test parameters through the correlation coefficient table; and S2034: defining the V original test parameters as eigen parameters.

As a multivariate statistical method for exploring into the correlation among a plurality of variables, principal component analysis is to study how the internal structures among a plurality of variables are revealed through a few principal components, i.e., a few principal components are derived from original variables, such that they reserve the information of the original variables as much as possible and are mutually uncorrelated. In general, mathematical processing means that the original P indicators are linearly combined as a new comprehensive indicator.

The principal component analysis in this embodiment generally includes the steps of:

(1) subjecting m final test parameters in each group (e.g., group 1) to standardization processing;

(2) calculating a correlation coefficient matrix;

(3) calculating eigen values and eigenvectors; and

(4) calculating % of variance and cumulative variance % of the eigen values, and obtaining the number of principal component variables according to the cumulative variance %.

In an embodiment, the process of determining a number P of principal component variables from the table of total variance explained includes: setting cumulative variation proportion thresholds; and based on the table of total variance explained of the principal components, when a cumulative variation proportion corresponding to a certain principal component is within a range of the cumulative variation proportion thresholds, taking the corresponding principal component as the principal component variable.

FIG. 7 illustrates, in an embodiment, the table of total variance explained of the principal components obtained after the principal component analysis is carried out on the corresponding several test parameters in each group. This table includes: Dim.1 to Dim.10, which are serial numbers for the principal components; eigen values, each representing one principal component and arranged in the table in a descending order; % of variance, indicative of the proportions of the eigen values; and cumulative variance %, i.e., the proportion of the first m large eigen values in total eigen values, and when their proportion exceeds a particular value, original sample data can be represented by the first m principal components, as long as cumulative variance % that is more than 70% is taken in general; for example, in this embodiment, the first three principal components are taken and cumulative variance % reaches 72%; hence, only the first three principal components need to be taken, and three principal components, i.e., Dim.1, Dim.2 and Dim.3, are considered as the principal component variables. It shall be noted that the table of total variance explained in FIG. 7 is an example only and shall not limit the protection scope of the present application.

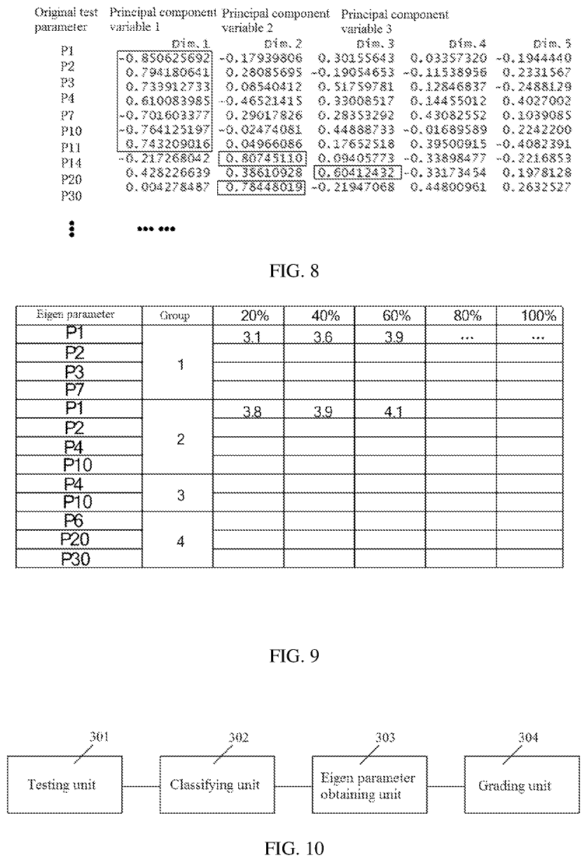

FIG. 8 illustrates, in an embodiment, the correlation coefficient table of the principal components and the test parameters obtained after the principal component analysis is carried out on the corresponding several test parameters in each group, i.e., the eigenvectors corresponding to the eigen values of FIG. 7 are found to create the table as illustrated in FIG. 8. As previously described, 3 principal component variables Dim.1, Dim.2 and Dim.3 are obtained, and the number V of the original test parameters are determined from the 3 principal component variables Dim.1, Dim.2 and Dim.3, through the correlation coefficient table. In particular, the number of the original test parameters is determined in sequence in accordance with the principal component variable 1-Dim.1, the principal component variable 2-Dim.2 and the principal component variable 3-Dim.3. First of all, in the case of the principal component variable 1-Dim.1, the eighth eigenvector suddenly decreases to 0.217268042 based on the magnitude of the eigenvectors, so the original parameters to which the principal component variable 1-Dim.1 is corresponding are the first seven parameters P1, P2, P3, P4, P7, P10 and P11; then, starting from the eighth original test parameter, in the case of the principal component variable 2-Dim.2, it is determined, based on the magnitude of the eigenvectors, that the original test parameters to which the principal component variable 2-Dim.2 is corresponding are the eighth original test parameter P14 and the tenth original test parameter P30; with the eigenvectors being higher than a preset value, the corresponding original test parameters P14 and P30 are obtained; next, in the case of the principal component variable 3-Dim.3, it is judged whether the remaining ninth original test parameter is selected as the original test parameter to which the principal component variable 3-Dim.3 is corresponding, and since the eigenvector to which the ninth original test parameter is corresponding satisfies the requirements, the principal component variable 3-Dim.3 corresponds to the original test parameter P20. For the 3 principal component variables Dim.1, Dim.2 and Dim.3, the number of the original test parameters is determined as 10 through the correlation coefficient table, i.e., P1, P2, P3, P4, P7, P10, P11, P14, P20 and P30. These 10 original test parameters are defined as eigen parameters in order to perform S204: determining, based on the eigen parameters, grades for the semiconductor products in each group.

The process of determining, based on the eigen parameters, grades for the semiconductor products in each group includes: determining the grades for the semiconductor products according to a percentage of the semiconductor products satisfying a preset condition, in the total quantity of the semiconductor products, wherein the preset condition is associated with the eigen parameters, and grading can be determined from the percentage and the eigen parameters. In particular, the grading process includes the following steps.

Grade standards are determined. The grade standards indicate an upper limit of a percentage of the semiconductor products satisfying the preset condition in the total quantity of the semiconductor products, under a particular eigen parameter. For example, the first grade means that under a particular eigen parameter, the percentage of the semiconductor products satisfying the preset condition in the total quantity of the semiconductor products is less than 20%, and the second grade means that under a particular eigen parameter, the percentage of the semiconductor products satisfying the preset condition in the total quantity of the semiconductor products is less than 40%, etc. Further, the span (i.e., grade distance)among the various grade standards should not be too large or too small, so as to avoid losing the significance of grading. In this embodiment, the span among the various grades is 20%, and preferably in other embodiments of the present application, the span among the various grades is 5% to 10%.

The percentage of the semiconductor products satisfying the preset condition in the total quantity of the semiconductor products under a particular eigen parameter is calculated. The preset condition may be that a numeral value corresponding to this eigen parameter is less than a preset numeral value. For instance, in a particular group, the percentage of the semiconductor products with a particular eigen parameter is less than the preset numeral value, in the total quantity of the semiconductor products.

The semiconductor products are graded according to the percentage and the grade standards. For example, in a particular group, the percentage of the semiconductor products with a particular eigen parameter less than the preset numeral value, in the total quantity of the semiconductor products is 8%, and then this group is classified into the first grade.

By way of example, with reference to FIG. 9, an eigen parameter P1 is present in both the group 1 and the group 2, and there are a plurality of numeral values for the eigen parameter P1, e.g., P1 is 3.1, 3.6, 3.8, 3.9 and 4.1. Then in the group 1, it can be known from calculations that the percentage of the semiconductor products with the eigen parameter P1 less than 3.1, in the total quantity of the semiconductor products is 20%; the percentage of the semiconductor products with the eigen parameter P1 less than 3.6, in the total quantity of the semiconductor products is 40%; and the percentage of the semiconductor products with the eigen parameter P1 less than 3.9, in the total quantity of the semiconductor products is 60%. On the basis of the preset grade standards, if, for the eigen parameter P1, the standard for the first grade is that the percentage of the semiconductor products with the eigen parameter P1 less than 3.6, in the total quantity of the semiconductor products is 40%, then the group 1 meets the standard for the first grade and is thus classified into the first grade; if, for the eigen parameter P1, the standard for the first grade is that the percentage of the semiconductor products with the eigen parameter P1 less than 3.6, in the total quantity of the semiconductor products is 60%, and the standard for the second grade is that the percentage of the semiconductor products with the eigen parameter P1 less than 3.6, in the total quantity of the semiconductor products is 40%, then the group 1 does not meet the standard for the first grade but meets the standard for the second grade, and is thus classified into the second grade; similarly, the group 2 and the like may also be graded according to the eigen parameter P1, and this is not described in detail here. In the above example, grading is carried out on the basis of the eigen parameter P1, while in other embodiments, grading may also be carried out on the basis of other eigen parameters, in order to satisfy different requirements.

It shall be noted that the step of classifying the several semiconductor products into a plurality of groups according to the several test parameters and the step of carrying out a principal component analysis on the corresponding several test parameters in each group to obtain eigen parameters for each group are performed on a distributed operation server, in order to increase the efficiency of data operation. The foregoing grading method of the present application includes: at the time of an electric property test, classifying the several semiconductor products into a plurality of groups according to several test parameters obtained from the test; carrying out a principal component analysis on the corresponding several test parameters in each group, to obtain eigen parameters for each group; and determining, based on the eigen parameters, grades for the semiconductor products in each group. The semiconductor products in each group are graded through several eigen parameters with similar attributes, so the accuracy of the existing semiconductor product grading is improved and the semiconductor products that meet different customer requirements can be accurately and quickly screened out for shipping.

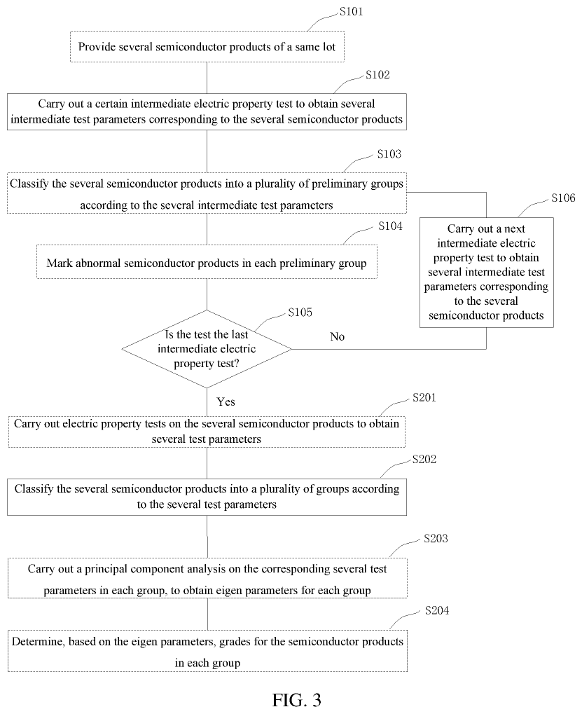

In the above embodiment, the electric property test is a final test, and before the final test is carried out, it is further required that a plurality of intermediate electric property tests are carried out in sequence. With reference to FIG. 3, in other embodiments, prior to S201 that the final test is carried out, it is further required that a plurality of intermediate electric property tests are carried out in sequence, and several intermediate test parameters corresponding to several semiconductor products are obtained from each intermediate electric property test. In particular, the following steps are included: S101: providing several semiconductor products of a same lot; S102: carrying out a certain intermediate electric property test to obtain several intermediate test parameters corresponding to the several semiconductor products; S103: classifying the several semiconductor products into a plurality of preliminary groups according to the several intermediate test parameters; and S104: marking abnormal semiconductor products in each preliminary group.

After S104 is performed, S105 is also included: judging whether the intermediate electric property test is the last intermediate electric property test; if “yes”, then S201 to S204 are performed in sequence; and if “not”, S106 is performed:

carrying out a next intermediate electric property test to obtain several intermediate test parameters corresponding to the several semiconductor products. And subsequent to S106, S103 to S105 are performed in sequence again.

Prior to the final test, multiple intermediate electric property tests need to be carried out. Most of the electric parameters to be tested between the final test and the intermediate electric property tests as well as between the different intermediate electric property tests are different, so certain property on the semiconductor product can be known only during a particular intermediate test. Hence, a test parameter recording for the semiconductor products can be created by classifying the semiconductor products into a plurality of preliminary groups through use of the foregoing method, such that the various parameters of the semiconductor products are learnt in a more accurate way, and thus reference can be made to these parameters whenever necessary, in order to accurately and rapidly screen out the semiconductor products that meet different customer requirements. Furthermore, the abnormal semiconductor products may be marked in a timely manner prior to the final test, which can help us understand the parameters through the marking and also facilitate supervision on the abnormal semiconductor products.

The method of classifying the several semiconductor products into a plurality of preliminary groups according to the several intermediate test parameters in S103 is the same as or similar to the method of classifying the several semiconductor products into a plurality of groups according to the several test parameters in the foregoing S202, and a description thereof is not given here.

In an embodiment, the process of marking abnormal semiconductor products in each preliminary group in S104 includes: providing an empirical database having a test parameter sequence of historical abnormal semiconductor products stored therein; matching the test parameter sequence of the semiconductor products in the preliminary group with the test parameter sequence of the historical abnormal semiconductor products in the empirical database; and if the both are matched with each other, considering the semiconductor products in the preliminary group as the abnormal semiconductor products, and marking the abnormal semiconductor products.

The empirical database has the test parameter sequence of the historical abnormal semiconductor products stored therein. The test parameter sequence of the historical abnormal semiconductor products is x=(par1, par2, par3, . . . , parN), wherein par1, par2, par3 . . . parN represents different test parameters, and the test parameter sequence of the semiconductor products in the preliminary group is y=(par1, par2, par3, . . . , parN); cos<x,y>=(x·y)/(|x∥y|) is calculated through x and y, wherein “·” is an inner product symbol and “∥” is a length symbol; if cos<x,y>≥ a set threshold (e.g., 0.9), then the test parameter sequence of the semiconductor products in the preliminary group is considered to be matched with the test parameter sequence of the historical abnormal semiconductor products in the empirical database, and the semiconductor products in the preliminary group are considered as abnormal semiconductor products and these abnormal semiconductor products are marked.

Also provided in an embodiment of the present application is a semiconductor product grading system, which, with reference to FIG. 10, includes:

a testing unit 301, configured to carry out electric property tests on several semiconductor products of a same lot, to obtain several test parameters; the electric property tests include a final test and intermediate electric property tests, and in this embodiment, the electric property test is a final test to obtain several final test parameters;

a classifying unit 302, configured to classify the several semiconductor products into a plurality of groups according to the several test parameters;

an eigen parameter obtaining unit 303, configured to carry out a principal component analysis on the corresponding several test parameters in each group, to obtain eigen parameters for each group; and

a grading unit 304, configured to determine, based on the eigen parameters, grades for the semiconductor products in each group.

In an embodiment, an algorithm used by the classifying unit 302 to classify the several semiconductor products into a plurality of groups according to the several test parameters is a clustering algorithm.

The clustering algorithm is a K-Means clustering algorithm, a mean shift clustering algorithm, a density-based clustering algorithm, a grid-based clustering algorithm, or a model-based clustering algorithm.

In an embodiment, the process of classifying several semiconductor products into a plurality of groups through use of the K-Means clustering algorithm includes: setting a plurality of different K values, and executing the K-Means clustering algorithm under different K values, to obtain a plurality of corresponding pending groups; obtaining a percentage of inter-group variations of the pending groups under different K values, in a total variation; judging whether a percentage of the inter-group variations corresponding to the pending groups under a certain K value, in the total variation, is within a set threshold range, and if the percentage is within the set threshold range, then taking K pending groups obtained under this K value, as the plurality of groups classified.

In an embodiment, a range for the plurality of different K values is 2 to 30.

In an embodiment, the process for the eigen parameter obtaining unit 303 to carry out a principal component analysis on the corresponding several test parameters in each group to obtain main eigen parameters for each group includes: carrying out the principal component analysis on the corresponding several test parameters in each group, to yield a correlation coefficient table of principal components and test parameters as well as a table of total variance explained of the principal components; determining a number P of principal component variables from the table of total variance explained; determining, for the P principal component variables, a number V of original test parameters through the correlation coefficient table; and defining the V original test parameters as eigen parameters.

The process of determining, based on the eigen parameters, grades for the semiconductor products in each group includes: determining the grades for the semiconductor products according to a percentage of the semiconductor products satisfying a preset condition, in the total quantity of the semiconductor products, the preset condition being associated with the eigen parameters.

In an embodiment, the testing unit 301 is configured not only to carry out the final test, but also to carry out, before carrying out the final test, a plurality of intermediate electric property tests in sequence, several intermediate test parameters corresponding to several semiconductor products being obtained from each intermediate electric property test; the classifying unit 302 is further configured to classify the several semiconductor products into a plurality of preliminary groups according to the several intermediate test parameters; the grading system further includes an abnormal semiconductor product marking unit and a removing unit, and the abnormal semiconductor product marking unit is configured to mark abnormal semiconductor products in each preliminary group.

In an embodiment, a judging unit is also included, which is configured to, after a certain intermediate electric property test is carried out and the abnormal wafers are marked, judge whether this intermediate electric property test is the last intermediate electric property test; if “yes”, the step of carrying out the final test on the several semiconductor products to obtain several final test parameters is performed by the testing unit; and if “not”, the step of carrying out a next intermediate electric property test to obtain several intermediate test parameters corresponding to the several semiconductor products is performed by the testing unit.

In an embodiment, the process for the abnormal semiconductor product marking unit to mark the abnormal semiconductor products includes: providing an empirical database having a test parameter sequence of historical abnormal semiconductor products stored therein; matching the test parameter sequence of the semiconductor products in the preliminary group with the test parameter sequence of the historical abnormal semiconductor products in the empirical database; and if the both are matched with each other, considering the semiconductor products in the preliminary group as the abnormal semiconductor products, and marking the abnormal semiconductor products.

In an embodiment, the classifying unit and the eigen parameter obtaining unit are located on a distributed operation server.

It shall be noted that the definition or description of the same or similar sections in this embodiment (grading system) as in the previous embodiment (grading process) will not be given in this embodiment. Reference is made to the definition or description of the corresponding sections in the previous embodiment.

It is to be understood that the above specific implementations of the present application are used only to exemplify or explain the principle of the present application, and do not constitute a limitation to the present application. Therefore, any modification, equivalent substitution, improvement, etc. made without departing from the spirit and scope of the present application shall be included in the protection scope of the present application. In addition, the appended claims of the present application are intended to cover all variation and modification examples that fall within the scope and boundary of the appended claims, or within the equivalent forms of such scope and boundary.

What is claimed is:

1. A semiconductor product grading method, comprising:
providing several semiconductor products of a same lot;
carrying out electric property tests on the several semiconductor products to obtain several test parameters;
classifying the several semiconductor products into a plurality of groups according to the several test parameters;
carrying out a principal component analysis on the corresponding several test parameters in each group, to obtain eigen parameters for each group; and
determining, based on the eigen parameters, grades for the semiconductor products in each group.

2. The semiconductor product grading method according to claim 1, wherein an algorithm used in the step of classifying the several semiconductor products into a plurality of groups according to the several test parameters is a clustering algorithm.

3. The semiconductor product grading method according to claim 1, wherein the process of carrying out a principal component analysis on the corresponding several test parameters in each group to obtain eigen parameters for each group comprises:
carrying out the principal component analysis on the corresponding several test parameters in each group, to obtain a correlation coefficient table of principal components and test parameters as well as a table of total variance explained of the principal components;
determining a number P of principal component variables from the table of total variance explained;
determining, for the P principal component variables, a number V of original test parameters through the correlation coefficient table; and
defining the V original test parameters as eigen parameters.

4. The semiconductor product grading method according to claim 1, wherein the process of determining, based on the eigen parameters, grades for the semiconductor products in each group comprises: determining the grades for the semiconductor products according to a percentage of the semiconductor products satisfying a preset condition, in a total quantity of the semiconductor products, the preset condition being associated with the eigen parameters.

5. The semiconductor product grading method according to claim 1, wherein the electric property test is a final test to obtain several final test parameters, and before the final test is carried out, the following steps are further required: a plurality of intermediate electric property tests are carried out in sequence, several intermediate test parameters corresponding to several semiconductor products are obtained from each intermediate electric property test; the several semiconductor products are classified into a plurality of preliminary groups according to the several intermediate test parameters; and abnormal semiconductor products in each preliminary group are marked.

6. The semiconductor product grading method according to claim 5, wherein after a certain intermediate electric property test is carried out and the abnormal semiconductor products in each preliminary group are marked, it is judged whether this intermediate electric property test is a last intermediate electric property test; if “yes”, the step of carrying out the final test on the several semiconductor products to obtain several final test parameters is performed; and if “not”, the step of carrying out a next intermediate electric property test to obtain several intermediate test parameters corresponding to the several semiconductor products is performed.

7. The semiconductor product grading method according to claim 5, wherein the process of marking abnormal semiconductor products in each preliminary group comprises: providing an empirical database having a test parameter sequence of historical abnormal semiconductor products stored therein; matching the test parameter sequence of the semiconductor products in the preliminary group with the test parameter sequence of the historical abnormal semiconductor products in the empirical database; and if the both are matched with each other, considering the semiconductor products in the preliminary group as the abnormal semiconductor products, and marking the abnormal semiconductor products.

8. A semiconductor product grading system, comprising:
a testing unit, configured to carry out electric property tests on several semiconductor products of a same lot, to obtain several test parameters;
a classifying unit, configured to classify the several semiconductor products into a plurality of groups according to the several test parameters;
an eigen parameter obtaining unit, configured to carry out a principal component analysis on the corresponding several test parameters in each group, to obtain eigen parameters for each group; and
a grading unit, configured to determine, based on the eigen parameters, grades for the semiconductor products in each group.

9. The semiconductor product grading system according to claim 8, wherein an algorithm used by the classifying unit to classify the several semiconductor products into a plurality of groups according to the several test parameters is a clustering algorithm.

10. The semiconductor product grading system according to claim 8, wherein the electric property test is a final test to obtain several final test parameters, and the testing unit, before carrying out the final test, is further configured to: carry out a plurality of intermediate electric property tests in sequence, several intermediate test parameters corresponding to several semiconductor products being obtained from each intermediate electric property test; the classifying unit is further configured to classify the several semiconductor products into a plurality of preliminary groups according to the several intermediate test parameters; further comprising an abnormal semiconductor product marking unit, which is configured to mark abnormal semiconductor products in each preliminary group.

11. The semiconductor product grading system according to claim 10, further comprising a judging unit, which is configured to, after a certain intermediate electric property test is carried out and the abnormal semiconductor products in each preliminary group are marked, judge whether this intermediate electric property test is a last intermediate electric property test; if “yes”, the step of carrying out the final test on the several semiconductor products to obtain several final test parameters is performed by the testing unit; and if “not”, the step of carrying out a next intermediate electric property test to obtain several intermediate test parameters corresponding to the several semiconductor products is performed by the testing unit.

12. The semiconductor product grading system according to claim 8, wherein the classifying unit and the eigen parameter obtaining unit are located on a distributed operation server.
